(19) United States
(12) Patent Application Publication
(10) Pub. No.: US 2024/0398262 A1
(43) Pub. Date: Dec. 5, 2024

(54) METHOD AND PRESSURE SENSOR UNIT

(71) Applicant: KARMA Technologies AG, St. Gallen (CH)

(72) Inventors: Danilo SANTAGATA, Wil (CH); Rijad ZUZO, St. Gallen (CH); Heini SEGER, Niederteufen (CH)

(73) Assignee: KARMA Technologies AG, St. Gallen (CH)

(21) Appl. No.: 18/697,273

(22) PCT Filed: Sep. 29, 2022
(86) PCT No.: PCT/EP2022/077107

§ 371 Date: Mar. 29, 2024

(30) Foreign Application Priority Data
Sep. 30, 2021 (EP) 21200161.4

(51) Int. Cl. A61B 5/11; A61B 5/00; A61B 5/103; G01L 9/00

(52) U.S. Cl. CPC A61B 5/1126; A61B 5/1036; A61B 5/6892; G01L 9/0072

(57) ABSTRACT

A pressure sensor sheet (3) comprises a first layer (31) with first conductor paths (310) forming rows of a matrix and a second layer (32) with second conductor paths (320) forming columns of a matrix. The matrix has intersection points (30) being separated by a central layer (33) and forming sensor points (30). The rows and columns are cyclically sampled by a counter unit in order to determine the values at all of the intersection points (30). An electric voltage is applied to a single conductor path of the first conductor paths (310) and changes in the second conductor paths (320) are individually determined one after the other. All of the changes determined on the second conductor paths (320) are converted by a single analog-digital converter (ADC). This is repeated until all the rows and all the columns of the matrix have been cyclically sampled.

### TECHNICAL AREA

The present invention relates to methods for querying sensor values of a pressure sensor sheet, in particular a textile pressure sensor sheet, and a pressure sensor unit.

### PRIOR ART

Optimum patient monitoring is an increasing problem in particular due to the understaffing of personnel in the care sector. Technical solutions can provide a remedy here and ensure more safety and relief in patient care.

U.S. Pat. No. 5,144,284 discloses a mat having pressure sensors which can be laid on the bed to monitor the movement of a patient. WO 2012/153263 A1 relates to a sensor cushion having pressure sensors for sleep monitoring.

WO 01/75924 A1 describes pressure sensors in the form of an electrically conductive fabric. It is mentioned that such a sensor fabric can be used in combination with a mattress. EP 1 269 502 B1 discloses a sensor mat having an electrically conductive layer.

EP 3 736 364 A1 discloses a textile sensor mat for a bed, wherein the sensor mat is used to determine the pressure distribution in bedridden people.

In EP 3 415 134 B1 the risks of bedsores of bedridden patients are to be monitored by means of a sensor mat and thus reduced. Strip-shaped sensors are used for this purpose. EP 3 769 676 A1 also monitors pressure changes on a bed by means of strip-shaped sensors.

EP 3 447 463 B1 from Sefar AG discloses a textile pressure sensor. In an embodiment according to FIGS. 7 and 8, a first fabric layer has electrically conductive threads which extend in a first direction. A second fabric layer having electrically conductive threads extends in a second direction which runs perpendicular to the first direction. A piezoresistive third fabric layer is arranged between the two fabric layers. This third fabric layer changes its resistance and thus its electrical conductivity depending on the pressure force action. If a measurement current is applied to the electrically conductive threads and the mat is subjected to a pressure, the electrical resistance changes at the intersection areas. This change is used as a measure of the applied pressure and can be acquired by a control unit.

J. Cheng, et al., Smart-surface: Large scale textile pressure sensors arrays for activity recognition, Pervasive and Mobile Computing (2016), http://dx.doi.org/10.1016/j.pmcj.2016.01.007, discloses a sensor mat from Sefar AG having a third fabric layer, which consists of a pressure-sensitive material having the trademark name CARBOTEX® of Sefar AG. The electrically conductive threads form a grid or a matrix n×m having intersection areas. Each intersection area acts as a pressure sensor and corresponds to one pixel of the pressure distribution matrix. An FPGA (Field Programmable Gate Array) controls ultrafast switch units (Switch Arrays) and analog-to-digital converters (ADC) and collects data of the matrix. Each matrix column i of n is switched on individually, while the others are deactivated. The voltages in the m lines then correspond to those at the intersection areas of the m lines of the ith column. These voltages of the m lines are passed on to multiplexers and fed into the ADCs. In the next step, the next column i+1 is switched on and the corresponding voltages in the m lines are passed on. How the individual multiplexers are controlled is not described. The column by column query results in a data series which corresponds to the pressure distribution. n ADCs and n associated multiplexers are required. It is specified that 128×128 sensor points in 40 images per second can be passed on to a computer. An image analysis is then carried out.

The more sensor points a sensor mat has, the more time is required for acquiring the measured values. This increases the costs for the corresponding electronics and software.

### DESCRIPTION OF THE INVENTION

It is an object of the invention to provide a method for querying sensor values of a pressure sensor sheet and a pressure sensor unit which can read out the sensor values of the entire sheet in the shortest possible time and are nonetheless cost-effective.

This object is achieved by a method and a pressure sensor unit having the features of claims 1 and 16, respectively.

The method according to the invention for querying sensor values of a pressure sensor sheet uses a pressure sensor sheet which comprises
a first layer having first conductor tracks extending parallel to one another,
a second layer having second conductor tracks extending parallel to one another, and
a middle layer arranged between the first and second layer.

The first and second layer are arranged relative to one another such that the first conductor tracks extend perpendicular to the second conductor tracks and the first and second layer form a common grid, matrix hereinafter. The matrix has lines, formed by the first conductor tracks, and columns, formed by the second conductor tracks.

The lines and the columns of the matrix form intersection points in which the first and second conductor tracks intersect spaced apart from one another by the middle layer and which are used as sensor points.

An external pressure action changes the distance between the first and second conductor tracks in at least one of the intersection points. This change is detected by means of an electronic circuit and read out as a value, wherein for the purpose of reading out the value an electric voltage is applied to the first conductor tracks and the change at the second conductor tracks is determined.

By means of at least one counter unit, the lines having the first conductor tracks and the columns having the second conductor tracks are cycled through to determine the values at all intersection points individually in succession. In this case
a) as provided by the at least one counter unit, a first switch unit is actuated and the electric voltage is applied to a single one of the first conductor tracks, and
b) as provided by the at least one counter unit, a second switch unit is repeatedly actuated and the changes in the second conductor tracks are individually determined in succession.

All changes determined on the second conductor tracks are converted by a single analog-to-digital converter (ADC). Steps a) and b) are repeated in that in step a) according to the at least one counter unit, the first switch unit is actuated to apply the electric voltage to a single other one of the first conductor tracks. Steps a) and b) are preferably repeated until all lines and all columns of the matrix have been cycled through.

The terms “lines” and “columns” are not restricted to the orientation of the sheet on a bed. They are also not restricted to the columns extending in the longitudinal direction and the lines extending in the transverse direction of the sheet. They are merely to represent that the first conductor tracks extend perpendicular to the second conductor tracks and the conductor tracks form a grid structure in the top view.

The term “sheets” not only comprises units which are laid on a bed. It also comprises units for use in, on, or at bodies which are used by the human body for lying or sitting. For example, they are part of a mattress, a bedframe, a lounger, an armchair, a chair, a sofa, or a car seat. The terms “pressure sensor sheet” and “sensor sheet” are used hereinafter. However, they also comprise the mentioned embodiments integrated in other bodies, laid on other bodies, or connected to other bodies.

The pressure sensor sheet is preferably a textile sensor sheet.

The layers are preferably, but not exclusively, fabric layers. The term “fabric layers” is used hereinafter, wherein other types of self-supporting and non-self-supporting material layers are also comprised. The layer is preferably self-supporting.

In some embodiments, the layers are material layers on which or in which the first and second conductor tracks are arranged. In other embodiments, one or both layers is or are formed by the conductor tracks themselves and do not consist of an independent, separate material layer. Thus, for example, the first and/or the second conductor tracks may be sewed onto the middle layer. For this purpose, for example, an embroidery machine may be used, which fastens the first or second conductor tracks in a predetermined pattern on the middle layer. An electrically nonconductive thread is preferably used for this purpose.

Preferably, only the first or the second conductor tracks are sewed onto the middle layer. The other conductor tracks are sewed onto an independent, separate material layer, in particular a textile material. The material is preferably a woven fabric, a warp-knitted fabric, a milled material, a knitted fabric, a mesh, a knitted mat, a nonwoven material, or a felt. In this embodiment, the flexprints mentioned below in the text are preferably fastened on this independent, separate material layer, preferably also sewed on. I.e., the flexprints, which are connected to the conductor tracks fastened to the middle layer, are also fastened on the independent, separate material layer. The middle layer is preferably made smaller than the independent, separate material layer so that the separate material layer protrudes over at least one edge of the middle layer. Connecting tracks which connect the first and second conductor tracks to the flexprints are also sewed onto the separate material layer. The flexprints, also called electronics units, may additionally or alternatively also be adhesively bonded, fastened using glue strips or adhesive strips, crimped on, or fastened in another manner. The connecting tracks are preferably the integral extensions of the first and second conductor tracks. This means that those conductor tracks which are sewed onto the middle layer are sewed at at least one of their ends onto the separate material layer. The middle layer of this embodiment is preferably Carbotex®, a material sold by Sefar AG. Other materials which serve as sensor elements may also be used. The first and second conductor tracks are preferably electrically conductive threads, wires, or cables. They are preferably flat or round in their cross-section. They are preferably silver-coated copper wires or other wires which have an optimum conductivity.

In the method according to the invention, preferably an electric voltage is only applied to a single line of the sensor sheet at a time, i.e., only a single line is energized. No electric voltage is applied to the other lines of the sensor sheet, i.e., they are not energized and have a zero potential. Therefore, only the values of this single line can be read out at the columns, those of the other lines cannot. Since the columns are queried individually, only the value of a single intersection point is transmitted in each case, although the other columns could also supply signals.

Since the controller determines which line the voltage is applied to and since this controller also determines which column is currently read out, the location information of the intersection point and thus of the read-out sensor is known. The location information thus does not have to be supplied as a further value to the controller. A single measurement circuit per intersection point is sufficient. This reduces the amount of data and increases the speed during the query.

Furthermore, it is advantageous that it is not necessary to wait for “settling” of a voltage. The voltage may be detected directly at the analog-to-digital converter (ADC) when the shunt is laid for the line and the column, i.e., when the respective intersection point is set.

“Switch arrays” are preferably used as the switch units. The switch units are preferably not multiplexers.

The voltage evaluation takes place via the single ADC. The ADC is preferably a 12 bit ADC. The ADC preferably transmits the digitized value via a bus system (SPI) to a Raspberry Pi.

No further complex components are necessary.

Preferably, the digital IO (input, output) are not reconfigured during a readout process of the matrix, but only before a renewed query of the matrix. The digital IO (input, output) are typically part of the Raspberry Pi.

The ADC is preferably an independent component which is formed separately from the voltage evaluation.

The measurement circuits including the switch and counter units are preferably arranged on flexprints. The ADC is preferably also arranged on one of the flexprints. Preferably, one flexprint is provided for the lines and one flexprint is provided for the columns. Two or more flexprints for the lines or columns may also be arranged one behind another and may be connected. The flexprints are preferably part of the sensor sheet and incorporated into the envelope of the sheet.

Depending on the size of the sensor sheet, it may be divided into multiple sections, wherein each section forms a partial sheet. For example, the head and the torso area of the bed sheet may be formed as a first sensor sheet and the hip and leg area of the patient as a second sensor sheet. These sheets may be housed in a common envelope, but are to be understood as two separate sheets according to the invention with respect to the data acquisition. The acquired data are in this case first brought together and evaluated in an external data processing unit.

The material or the fabric between the conductor tracks of the individual columns is made electrically nonconductive so that these conductor tracks are galvanically separated from one another. This also applies to the material or the fabric between the conductor tracks of the individual lines. The middle layer is preferably a dielectric material or is electrically insulating.

The resistance changes of the second conductor tracks are typically measured. Known measurement circuits are used for this purpose. The analog-to-digital converter changes the analog signal into a digital signal.

Software preferably specifies the cycle for cycling through the lines and columns. One switch of the switch units is preferably actuated during each cycle. One switch of the first switch unit of a line is preferably left at “one” here, i.e., the voltage remains applied to the corresponding first conductor track until all columns have been cycled through by means of the second switch unit.

Preferably, adjacent columns and/or lines are cycled through in chronological succession, i.e., the next following second conductor track or the next following first conductor track is always activated. The controller preferably jumps one line further when all columns have been cycled through in the preceding line.

In other variants of the method, the lines and/or columns are cycled through in a predetermined sequence which differs from the sequence of the arrangement of the lines and/or the columns. This may be implemented using a single ADC or using multiple ADCs. In the case of the use of a single ADC, the individual conductor tracks are connected in hardware accordingly to the electronics or the connection between the at least one counter unit and the switch units is selected accordingly. Alternatively, the same connections are provided in hardware as in the case of incremental, successive querying, but the software does not control the switches incrementally, but rather in a predetermined pattern.

Preferably, before all lines and all columns of the matrix are cycled through again, the first and second switch unit are actuated such that an electrical voltage is not applied to any of the lines and none of the columns is connected to the analog-to-digital converter (ADC).

A DC voltage is preferably applied, preferably of approximately 3.3 V or 5 V. This minimizes the electromagnetic influencing of the patient.

The lines and/or the columns are preferably cycled through at a frequency of at least 16 MHz. A high cycle frequency increases the query speed.

The read-out values can be stored in different ways. The read-out values of all intersection points are preferably stored as a common value packet. Subsequent readouts are preferably stored as separate further common value packets. These value packets are preferably transmitted to a data processing unit.

The measured values are preferably stored in a data processing unit. This enables the evaluation of the values for later use, for example, for a prognosis of the behavior of the patient in bed. The prognosis can be used for early detection of health problems or medical emergencies or also to detect early whether the patient is about to get or fall out of bed. The stored values are preferably processed in a self-learning algorithm for prognoses of patient monitoring. Earlier data are compared to newly obtained data, any interacting actions of the care personnel which have been input into the system are taken into consideration, and depending on the embodiment, data from further patient monitoring actions using identical or similar systems from databases are also taken into consideration. The consideration is preferably weighted depending on the source and/or age of the data. Statistical mean values are preferably determined and used as the basis for further calculations.

The first switch unit preferably has multiple switch arrays and the second switch unit also has multiple switch arrays.

In preferred embodiments, at least one sensor is provided, preferably a temperature sensor or a moisture sensor, the sensor values of which are determined by means of the second switch unit according to the first and second counter unit. The sensor values are determined during the cycling through of the lines and columns as values of a line and column analogous to the values of the intersection points. The sensor is preferably connected to the corresponding electronics unit, preferably the corresponding flexprint.

This enables the additional acquisition of sensor values using the same matrix system, the same counter unit, and the same switch units.

The type of the query of the values does not have to be changed. The sensor values are read out again each time the individual columns are cycled through.

The values received from the at least one sensor are stored like the pressure sensor values in the common value packet. They are not specially handled. This facilitates the query, which is thus not delayed.

The entire value packet, i.e., including the sensor data, is preferably transmitted to the data processing unit. It is preferably recognized in the data processing unit on the basis of the line identification which values are to be assigned to which sensor. Preferably, one mean value per sensor is formed by a data processing unit from the multiple data following in close chronological succession of the at least one sensor.

The method according to the invention is therefore preferably used for monitoring of a patient lying in a bed, wherein the pressure sensor sheet is arranged on the bed and wherein the values are transmitted to a central or mobile patient monitoring unit.

A pressure sensor unit according to the invention for querying sensor values of a pressure sensor sheet uses a pressure sensor sheet which comprises
a first layer having first conductor tracks extending parallel to one another,
a second layer having second conductor tracks extending parallel to one another, and
a middle layer arranged between the first and second layer.

The first and second layer are arranged relative to one another such that the first conductor tracks extend perpendicular to the second conductor tracks and the first and second layer form a common grid, matrix hereinafter. The matrix has lines, formed by the first conductor tracks, and columns, formed by the second conductor tracks.

The lines and columns of the matrix form intersection points in which the first and second conductor tracks intersect spaced apart from one another by the middle layer and which are used as sensor points.

An external pressure action changes the distance between the first and second conductor tracks in at least one of the intersection points. This change is detectable by means of an electronic circuit and can be read out as a value, wherein for the purpose of reading out the value an electric voltage is applicable to the first conductor tracks and the change at the second conductor tracks is determinable.

The pressure sensor unit comprises
at least one counter unit for cycling through the lines having the first conductor tracks and for cycling through the columns having the second conductor tracks to read out the values at all intersection points,
at least one first switch unit for applying the electric voltage to the first conductor tracks, and
at least one second switch unit for determining the change at the second conductor tracks.

The pressure sensor unit comprises a single analog-to-digital converter (ADC) for converting the changes determined at the second conductor tracks into the values. By means of the at least one first switch unit and according to the at least one counter unit, the electric voltage is applicable in succession to the first conductor tracks, wherein the electric voltage is only applied in each case to a single one of the first conductor tracks. By means of the at least one second switch unit and according to the at least one counter unit, upon application of the electric voltage to one of the first conductor tracks, the changes of the second conductor tracks are each determinable individually in succession. The determined changes of all second conductor tracks are convertible in the single analog-to-digital converter (ADC).

This pressure sensor unit may be produced cost-effectively. It has a minimum of electronic components. Moreover, the measurement circuit may be arranged on flexprints. Flexprints are flexible electronic components. They are well known in the prior art. The carrier typically consists of plastic, the electrical connections are typically printed on.

The at least one counter unit is preferably software-controlled.

Preferably, the at least one counter unit and the at least one first and/or second switch unit are arranged on a flexprint or a series of flexprints connected to one another.

The pressure sensor sheet preferably has at least one sensor, preferably a temperature sensor or humidity sensor, and wherein the pressure sensor unit determines sensor data of the at least one sensor.

Preferably, two counter units are provided, wherein a first counter unit of these counter units cycles through the lines and a second counter unit of these counter units cycles through the columns.

Further embodiments are specified in the dependent claims.

### BRIEF DESCRIPTION OF THE DRAWINGS

A preferred embodiment of the invention is described hereinafter on the basis of the drawings, which serve solely for explanation and are not to be interpreted as restrictive. In the figures of the drawings:

FIG. 1 shows a schematic representation of a patient bed having a pressure sensor sheet according to the invention of a pressure sensor system according to the invention;

FIG. 2 shows a schematic representation of the pressure sensor system according to the invention;

FIG. 3 shows a detail of a layer of the pressure sensor sheet according to FIG. 1;

FIG. 4 shows a schematic exploded view of the pressure sensor sheet according to FIG. 1 without surrounding envelope;

FIG. 5 shows a schematic exploded view of the pressure sensor sheet according to FIG. 1 with surrounding envelope;

FIG. 6 shows a schematic exploded view of the pressure sensor sheet according to FIG. 1 with flexible electronic prints and without surrounding envelope;

FIG. 7 shows a cross-section through the pressure sensor sheet according to FIG. 1 without surrounding envelope, in a schematic representation;

FIG. 8 shows a schematic representation of intersecting conductor tracks of two layers of the pressure sensor sheet according to FIG. 1;

FIG. 9 shows a schematic representation of the pressure sensor sheet according to FIG. 1 with intersecting conductor tracks;

FIG. 10 shows a schematic representation of the system architecture of the readout unit of the pressure sensor sheet according to FIG. 1;

FIG. 11 shows a further schematic representation of the system architecture of the readout unit of the pressure sensor sheet according to FIG. 1, and

FIG. 12 shows a flow chart of the control of the pressure sensor sheet.

### DESCRIPTION OF PREFERRED EMBODIMENTS

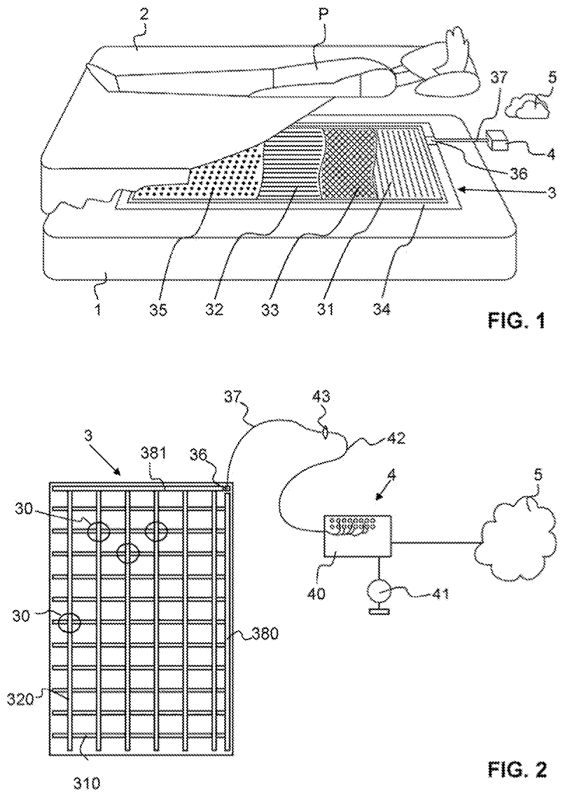

FIG. 1 schematically shows a patient bed having a pressure sensor system according to the invention.

A mattress 1 is covered as usual using a bedsheet 2, on which a patient P lies. A pressure sensor sheet 3 according to the invention is laid between mattress 1 and bedsheet 2. The pressure sheet 3 may be designed as a fitted sheet or, as shown here, as a planar flexible and preferably soft mat. It extends depending on the embodiment over the entire surface of the mattress 1 or ends, as shown here, spaced apart from the mattress edge. In other embodiments, the pressure sensor sheet 3 only extends in a partial area of the mattress 1, for example, only in the upper or in the middle or in the lower area of the mattress surface. In further embodiments, the pressure sensor sheet 3 is part of the mattress 1 or the bedsheet 2.

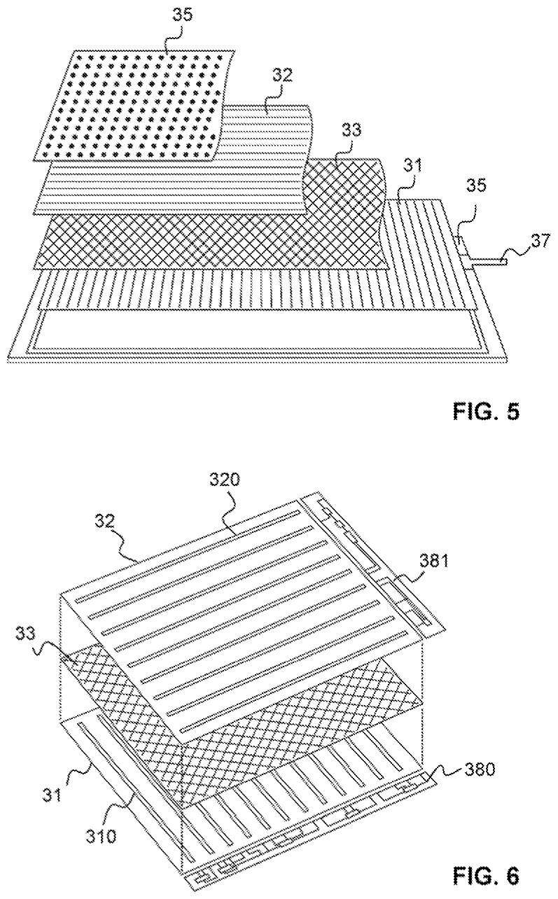

As can be seen well in FIGS. 1 and 5, the pressure sensor sheet 3 has a first layer or fabric layer 31, a second layer or fabric layer 32, and a middle layer 33 arranged in between. The three layers 31, 32, 33 are preferably connected to one another at points, preferably adhesively bonded. They are preferably jointly enclosed by a protective envelope, wherein the envelope preferably has a lower layer 34 and an upper layer 35, which depending on the embodiment consist of the same material or of different materials. They are preferably made soft and flexible, in particular a textile fabric or another suitable material layer. At least the upper one of the two layers 34, 35 is preferably provided with an incontinence protection, so that the three layers 31, 32, 33 in between are protected from moisture.

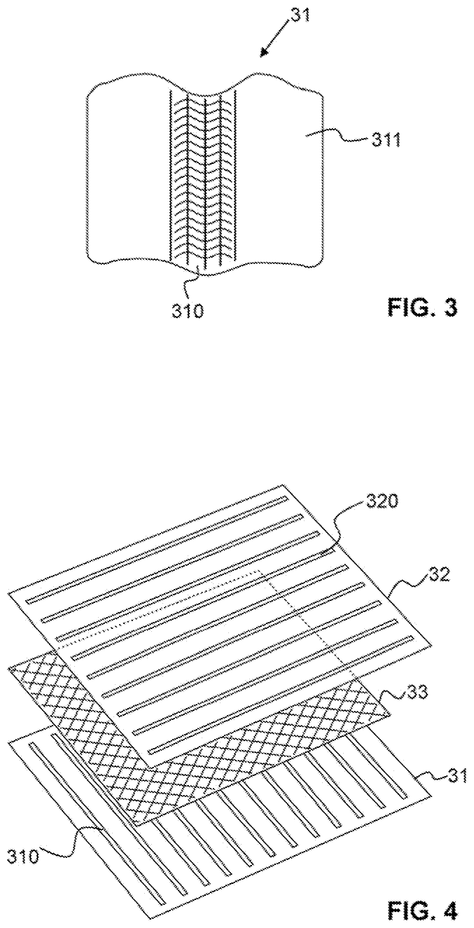

The first and second fabric layer 31, 32 are preferably manufactured from the same material. They have, as is shown on the basis of a detail according to FIG. 3 and in FIG. 4, multiple electrically conductive strips or tracks 310, 320 extending parallel to one another, which are preferably distributed over the entire or nearly the entire surface of the fabric layer 31, 32. They are preferably arranged distributed uniformly. The areas 311, 321 between the conductor tracks 310, 320 are made electrically nonconductive. The conductor tracks 310, 320 are preferably electrically conductive threads, which are woven with the nonconductive material of the fabric 311, 321. Such fabric layers 31, 32 are known from the prior art. Sensor fabrics as offered by Sefar AG are preferably used. These are mentioned, for example, in EP 3 447 463 B1, cited at the outset, and J. Cheng, et al., Smart-surface: Large scale textile pressure sensors arrays for activity recognition, Pervasive and Mobile Computing (2016), http://dx.doi.org/10.1016/j.pmcj.2016.01.007.

The middle layer 33 is manufactured from a pressure-sensitive, preferably dielectric material. The middle layer is preferably made homogeneous. It preferably consists of a material which changes its thickness proportionally in the range of the externally acting pressure to be expected and thus changes its electrical conductivity proportionally to the exerted pressure. The middle layer 33 preferably consists of Carbotex®, a material sold by Sefar AG. Other materials used as sensor elements may also be employed.

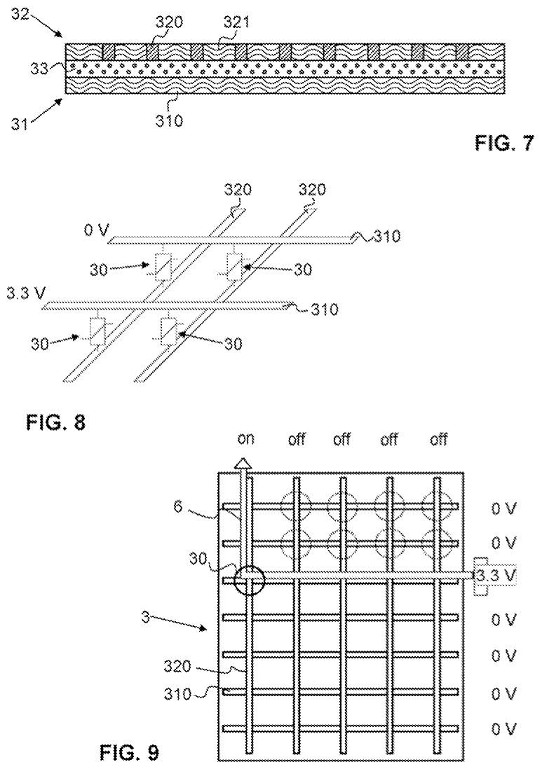

As can be seen in FIG. 4, the first and second fabric layer 31, 32 are laid one on top of another such that their conductor tracks 310, 320 have different orientations. The first conductor tracks 310 extend perpendicularly to the second conductor tracks 320. Intersection areas 30 thus result, as can be seen in FIG. 2. However, the conductor tracks 310, 320 do not contact one another in the intersection areas 30, since they are separated from one another by the middle layer 33. This can be seen in FIG. 7. The intersection areas 30 thus act as capacitors or resistors, as shown in FIG. 8. The capacitance of a capacitor changes in indirect proportion to the distance of its two capacitor plates. The capacitance at an intersection area 30 thus changes when a person rests on this intersection area 30 and thus compresses the middle layer 33.

The capacitance change results in a change of the resistance, which can be detected by means of a voltage divider. For this purpose, a constant DC voltage is applied to the second conductor tracks 320. The voltage is preferably 3.3 V or 5 V and thus does not influence the well-being of the patient.

Two strip-shaped, flexible electronic flexprints 380, 381 are also arranged in the envelope 34, 35 of the pressure sensor sheet 3. They are preferably fastened on the edges of the lower and upper layer 34, 35 protruding from the fabric layers 31, 32, 33, preferably adhesively bonded. A first flexprint strip 380 extends perpendicularly to the conductor tracks of the first fabric layer 31 and contacts all ends of the first conductor tracks 310 lying on one side of the fabric layer 31. A second flexprint strip 381 extends perpendicularly to the conductor tracks of the first fabric layer 32 and contacts all ends of the first conductor tracks 320 lying on one side of the fabric layer 32. The two flexprint strips 380, 381 thus extend perpendicularly to one another. This can be seen in FIG. 6. The flexprint strips 380, 381 are preferably modularly constructed and each consist of multiple sections, which are connected to one another. Each section typically has approximately 16 connections to the fabric layers. The flexprint strips thus form flexprint units.

The pressure sensor sheet 3 is connected via a connection 36 and a cable 37, preferably a 6-pole cable, to a control device 4. A control unit 40 of the control device is connected to a DC voltage source 41. Furthermore, it is connected to a data storage unit and/or a data evaluation unit. These can be arranged in one unit on location and/or can be cloud-based.

A cloud is provided with the reference sign 5 in FIG. 2. A plug connection of the cable 37 of the pressure sensor sheet 3 to the cable 42 of the control device 4 is symbolically shown and bears the reference sign 43. It is preferably a USB-C connection. The control device 4 is implemented as an SPI master, preferably on a Raspberry Pi.

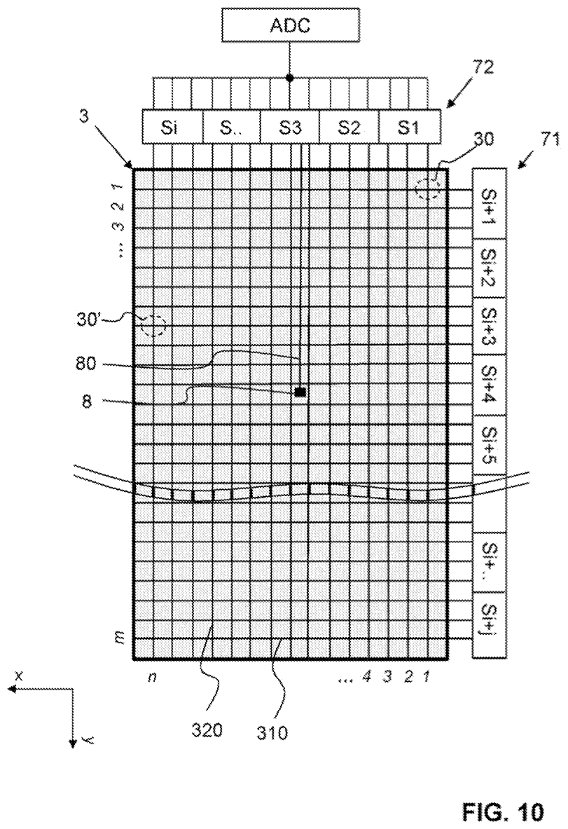

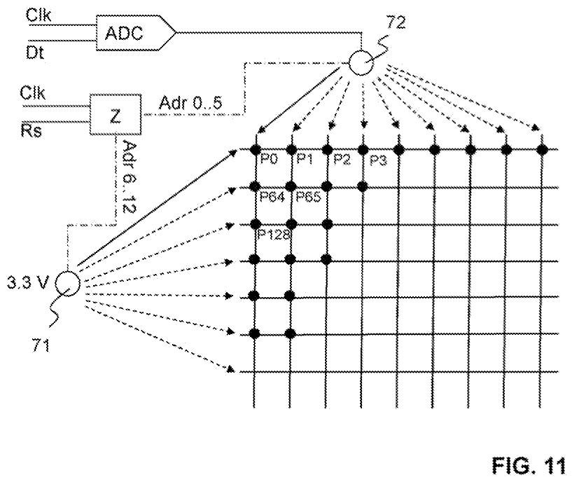

The mode of operation of the pressure sensor system according to the invention may be explained on the basis of FIGS. 9 to 12. As shown in FIG. 10, one switch unit 71, 72 in the form of multiple switch arrays S1, S2, Si; Si+1, . . . , Si+j is assigned in each case to the first conductor tracks 310 or the second conductor tracks 320 and connected thereto. Each switch array preferably has 16 connections for connection to the conductor tracks 310, 320. FIGS. 10 and 11 are therefore to be understood solely as schematic representations.

As can be seen in FIG. 9, each intersection area 30, i.e., each sensor, is individually queried. The electric voltage is applied for this purpose to only one line, i.e., a first conductor track 310, in each case, the values of all columns, i.e., the second conductor tracks 320, are read out in succession and only then is the next line “energized”, i.e., the electric voltage is applied. The remaining lines or first conductor tracks 310 are set to 0 V. A pulldown resistor is preferably provided for this purpose, which sets the other first conductor tracks 310 to 0 V. Accordingly, all lines are cycled through in succession.

In order that only a single intersection area 30 is detected, moreover all columns, i.e., second conductor tracks 320, except for one are set to inactive. I.e., the second switch unit 72 also opens the connection from the analog-to-digital converter ADC to a single second conductor track 320 here.

The active track 6 for detecting the intersection area 30 circled in bold is shown by an arrow in FIG. 9.

The value generated by the voltage divider, which is a measure of the pressure change, is changed in the analog-to-digital converter ADC into a digital signal and provided via the SPI bus to the SPI master, for example, the Raspberry Pi. The data may be transmitted via software interfaces, preferably via APIs (application programming interface) to higher-order software of a data storage unit and/or data evaluation unit. Measurements which supply high or low values in an above-average manner may be compensated for and filtered by software by means of typical methods. The read-out values may be smoothed by means of typical statistical methods.

In the example according to FIG. 10, therefore n columns and m lines are provided, which form an n×m matrix. A second switch unit 72 having i switches S1 . . . . Si, is assigned to the n columns, so that each switch can operate multiple columns. A first switch unit 71 having j−i switches Si+1, . . . . Si+j is assigned to the m columns, so that each of these switches can operate multiple of the m lines. The second switch unit 72 enables the connection between the second conductor tracks 320 and the ADC. Both switch units 71, 72 are connected via at least one counter unit Z to the controller. This is shown in FIG. 11. It can also be seen in FIG. 11 that via the first switch unit 71, as provided by the counter unit Z, a DC voltage can be applied to one of the first conductor tracks 310 and via the second switch unit 72, as provided by the counter unit Z, the change at the intersection points P0, P1, P2, can be transmitted to the ADC. CLK means clock, i.e., the clock cycle for the counters for switching the switches and for the ADC for reading out the values. Dt means data and shows that the ADC values can be transmitted to the external data storage unit or data processing unit. Rs means reset and shows that the counter can be set to zero again after querying all intersection points of the sensor map. In this example, the addresses Adr 0 to 5 are assigned to the columns having the second conductor tracks and the addresses Adr 6 to 12 are assigned to the lines having the first conductor tracks. This is only by way of example. A different number of addresses may also be used. The addresses are transferred in series using the clock to the two counter units. The addresses are preferably each increased by 1 using the clock and the electronics unit activates the next address. The two counter units thus result in the coordinates of the pressure point to be measured. On the basis of the count, which is incremented by the clocks, the software knows at each point in time which coordinates have to be assigned to the next sensor value.

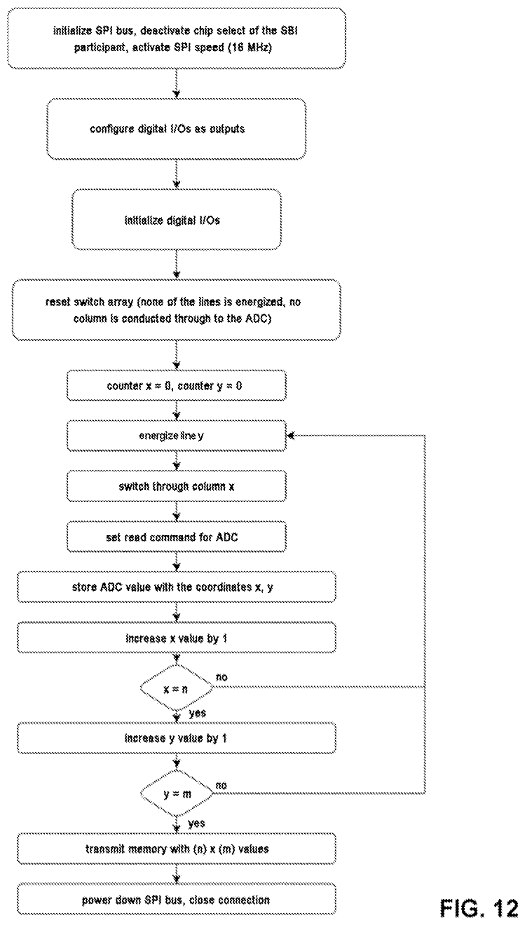

FIG. 12 shows a flow chart of the method according to the invention. The querying of all intersection points of the sensor mat is shown. The SPI bus is initialized in a first step.

In the next steps, the digital IO at the Raspberry Pi are configured and initialized. Furthermore, the switch units having their switch arrays are reset, so that the connections from the ADC to the second conductor tracks by means of the switch units are closed and the voltage at the first conductor tracks is set to 0 V. The counter units are moreover set to 0 for the columns x and the lines y.

The DC voltage is now applied at the first of the first conductor tracks 310. This is stated in the flow chart according to FIG. 12 in the step “energize line y”. The column x is then switched through, i.e., the ADC is connected to the second conductor track 320 of the column x. The value is read out by the ADC, converted into a digital signal, and stored with the coordinates x, y.

The sequence starts here with y=0 and x=0. This corresponds to the measurement point P0 in FIG. 11. The x value is then increased by 1 as indicated in FIG. 12. The columns are thus switched through in succession until x=n. In FIG. 12, n=64. The y value is now increased, due to which the DC voltage is applied to the next first conductor track 310 and the previously addressed first conductor track 310 is preferably set to 0 V. If y=m has been reached and therefore all intersection points have been read out, the memory having the n by m values is transmitted. The transmission of the matrix preferably takes place passed on to an internal service of the evaluation unit. This service preferably displays the values at a GUI (graphical user interface). Furthermore, the matrix is preferably passed on into a cloud.

Preferably, the SPI bus is powered down after each query of the matrix and the connections are closed by means of the switches. Upon a renewed query, which typically takes place within a few seconds or minutes, the SPI bus is reinitialized and the method begins again at the first step according to FIG. 12.

Thanks to the combination according to the invention of counter unit, switch units, and single ADC, no further multiplexers or further ADCs are necessary. The software, i.e., the controller, controls both switch units. It is thus already known which x and y coordinates are presently being measured in the n×m matrix, i.e., which intersection area 30. Which intersection area is currently being measured is therefore not determined and recognized via a measurement circuit, but rather specified via the software. This simplifies the electronics and increases the amount of data per unit of time. Furthermore, only one measurement circuit is necessary per intersection area 30.

The method according to the invention may be designed, for example, for sensor mats having 64 columns and 128 lines. Mats having 55 columns and 80 lines, i.e., having 4440 sensor points, may be read out completely within approximately 100 to 200 ms, for example.

Other dimensions for n and m are possible. The dimensions are dependent on the area of the pressure sensor sheet and on the resolution of the pressure measurement, i.e., on the number and the distances of the conductor tracks 310, 320 arranged parallel to one another. The clock frequency can also be different.

Furthermore, it is advantageous that the measurement circuits having the switch units and the ADC may be attached directly to the flexprints 380, 381. They are therefore located in the pressure sensor sheet 3. This facilitates the cable connections, facilitates the use on location, and enables a more flexible combination with external devices.

Furthermore, it is advantageous that the flexprint sections for the individual columns and lines can hardly be distinguished from one another. One flexprint has the ADC. The equipping of the flexprints differs in each case in which sections can be used. This is preferably defined by corresponding arrangement of 0 ohm resistors. The flexprints for the columns and lines also hardly differ from one another. This minimizes the production costs.

Furthermore, it is not necessary to wait until a voltage has “settled”. As soon as the shunt is set for the x and y axis of the n×m matrix, the voltage applied at the respective ADC may be directly measured. The digital IOs are preferably not reconfigured during a query of all n×m values. The SPI bus is preferably designed for read and write commands. A command may thus be transmitted to the respective ADC to transfer the sensor value and the sensor value is received as a response. This increases the speed of the query.

Furthermore, it is advantageous that values for a specific point in time are preferably determined and not average values. This increases the quality of the amount of data and is helpful in particular for the use of the data for early recognition, which is mentioned below in the text.

The obtained measured values of the individual intersection points result in information about where the patient lies, which points he or she loads more than others, and how he or she moves on the bed depending on the time.

Sleep behavior, pressure points, and in particular bedsore risks may thus be detected. It may also be detected whether a patient has left the bed.

One goal of the data collection is, however, early detection as to whether a patient suffering from dementia will leave the bed soon and/or whether the patient runs the risk of falling out of the bed. This is achieved in that data are collected and inferences are drawn from earlier behavior and measured values of patients in general and/or a specific monitored patient about his or her behavior upon leaving or upon falling out of the bed. It may thus be detected early whether a patient runs the risk of leaving or falling out of the bed unobserved. This early detection system can thus warn care personnel and trigger an alarm so that interventions can be taken as early as possible. As early as possible means if possible before the undesired event occurs.

Other undesired events, in which a patient predominantly behaves in the bed according to a specific scheme, can also be detected early in this way, for example, behavior in the case of increasing shortness of breath, epilepsy attacks, delirious patients, or in emergencies in which the care personnel or even an emergency team has to be on location as quickly as possible can be detected as early as possible thanks to these measurements and a self-learning data evaluation.

In FIG. 10, furthermore a sensor 8, for example, a temperature sensor, a moisture sensor, or another type of sensor, is connected to the second switch unit 72. The cable or the line is provided with the reference sign 80. The line 80 is preferably sewed on, glued on, or woven in. The readout behavior of this sensor does not differ from the readout behavior of the first and second conductor tracks. Preferably, the same electric voltage is applied. In other embodiments, a different electric voltage is applied in order to determine the sensor values. The method is explained in more detail in the section “description of the invention”. During the cycling through of the lines and columns as shown in the flowchart according to FIG. 12, one line is assigned to the sensor and the sensor values are read out when running through the columns for this line. If multiple sensors are provided, one line is assigned to each sensor.

The method according to the invention and the pressure sensor unit according to the invention enable patient monitoring which relieves the care personnel.

LIST OF REFERENCE SIGNS

| | | | |
|---|---|---|---|
| 1 | mattress | 4 | control device |
| | | 40 | control unit |
| 2 | bedsheet | 41 | DC voltage source |
| | | 42 | cable |
| 3 | pressure sensor sheet | 43 | plug |
| | pressure sensor matrix | | |
| 30 | intersection area | 5 | cloud |
| 31 | first fabric layer | | |
| 310 | first conductor track | 6 | active track of the measured |
| 311 | first nonconductive area | | value collection |
| 32 | second fabric layer | | |
| 320 | second conductor track | 71 | first switch unit |
| 321 | second nonconductive area | 72 | second switch unit |
| 33 | middle layer | | |
| 34 | lower layer | 8 | sensor |
| 35 | upper layer | 80 | line |
| 36 | connection | | |
| 37 | cable | P | patient |
| 380 | first flexprint | ADC | analog-to-digital converter |
| 381 | second flexprint | Z | counter unit |

1. A method for querying sensor values of a pressure sensor sheet, wherein the pressure sensor sheet comprises
a first layer having first conductor tracks extending parallel to one another,
a second layer having second conductor tracks extending parallel to one another, and
a middle layer arranged between the first and second layer,
wherein the first and second layer are arranged relative to one another such that the first conductor tracks extend perpendicular to the second conductor tracks, and the first and second layer form a common matrix, having lines, formed by the first conductor tracks, and having columns, formed by the second conductor tracks,
wherein the lines and the columns of the matrix form intersection points, in which the first and second conductor tracks intersect spaced apart from one another by the middle layer and which are used as sensor points,
wherein an external pressure action changes the distance between the first and second conductor tracks in at least one of the intersection points and wherein this change is detected by means of an electronic circuit and read out as a value, wherein for the purpose of reading out the value, an electric voltage is applied to the first conductor tracks and the change is determined on the second conductor tracks
wherein
by means of at least one counter unit, the lines having the first conductor tracks and the columns having the second conductor tracks are cycled through to determine the values at all intersection points individually in succession,
a) a first switch unit is actuated as provided by the at least one counter unit and the electric voltage is applied to a single one of the first conductor tracks,
b) a second switch unit is repeatedly actuated as provided by the at least one counter unit and the changes in the second conductor tracks are individually determined in succession, wherein all changes determined on the second conductor tracks are converted by a single analog-to-digital converter, and
steps a) and b) are repeated in that in step a), the first switch unit is actuated as provided by the at least one counter unit in order to apply the electric voltage to a single different one of the first conductor tracks, wherein steps a) and b) are repeated until all lines and all columns of the matrix have been cycled through.

2. The method as claimed in claim 1, wherein adjacent columns are cycled through in chronological succession.

3. The method as claimed in claim 1, wherein adjacent lines are cycled through in chronological succession.

4. The method as claimed in claim 1, wherein the lines and/or the columns are cycled through in a predetermined sequence which differs from the sequence of the arrangement of the lines and/or the columns.

5. The method as claimed in claim 1, wherein before all lines and all columns of the matrix are cycled through again, the first and second switch unit are actuated such that an electric voltage is not applied to any of the lines and none of the columns is connected to the analog-to-digital converter (ADC).

6. The method as claimed in claim 1, wherein a DC voltage is applied, preferably of approximately 3.3 V or 5 V.

7. The method as claimed in claim 1, wherein the lines and/or the columns are cycled through at a frequency of at least 16 MHz.

8. The method as claimed in claim 1, wherein the read-out values of all intersection points are stored as a common value packet.

9. The method as claimed in claim 8, wherein the value packet is transmitted to a data processing unit.

10. The method as claimed in claim 8, wherein the stored values are processed in a self-learning algorithm for prognoses of a patient monitoring action.

11. The method as claimed in claim 1, wherein the first switch unit comprises multiple switch arrays and wherein the second switch unit comprises multiple switch arrays.

12. The method as claimed in claim 1, wherein at least one sensor, preferably a temperature sensor or a moisture sensor, is provided, the sensor values of which are determined by means of the second switch unit as provided by the first and second counter unit, wherein the sensor values are determined during the cycling through of the lines and columns as values of a line and column analogously to the values of the intersection points.

13. The method as claim 12, wherein the read-out values of all intersection points are stored as a common value packet and wherein the sensor values are stored in the common value packet.

14. The method as claimed in claim 13, wherein the value packet is transmitted to a data processing unit and wherein the value packet is transmitted to the data processing unit.

15. A method for monitoring a patient lying in a bed, comprising carrying out the method of claim 1, wherein the pressure sensor sheet is arranged on the bed, and transmitting the values to a central or mobile patient monitoring unit.

16. A pressure sensor unit for querying sensor values of a pressure sensor sheet, wherein the pressure sensor sheet comprises
a first layer having first conductor tracks extending parallel to one another,
a second layer having second conductor tracks extending parallel to one another, and
a middle layer arranged between the first and second layer,
wherein the first and second layer are arranged relative to one another such that the first conductor tracks extend perpendicular to the second conductor tracks, and the first and second layer form a common matrix, having lines, formed by the first conductor tracks, and having columns, formed by the second conductor tracks,
wherein the lines and the columns of the matrix form intersection points, in which the first and second conductor tracks intersect spaced apart from one another by the middle layer and which are used as sensor points,
wherein an external pressure action changes the distance between the first and second conductor tracks in at least one of the intersection points and wherein this change is detectable by means of an electronic circuit and able to be read out as a value, wherein for the purpose of reading out the value, an electric voltage is able to be applied to the first conductor tracks and the change is determinable on the second conductor tracks
wherein
the pressure sensor unit comprises at least one counter unit for cycling through the lines having the first conductor tracks and for cycling through the columns having the second conductor tracks to read out the values at all intersection points,
the pressure sensor unit comprises at least one first switch unit for applying the electric voltage to the first conductor tracks,
the pressure sensor unit comprises at least one second switch unit for determining the change at the second conductor tracks,
the pressure sensor unit comprises a single analog-to-digital converter for converting the changes determined at the second conductor tracks into the values,
by means of the at least one first switch unit and as provided by the at least one counter unit, the electric voltage is applicable in succession to the first conductor tracks, wherein the electric voltage is applied in each case to only a single one of the first conductor tracks,
by means of the at least one second switch unit and as provided by the at least one counter unit, upon application of the electric voltage to one of the first conductor tracks, in each case the changes of the second conductor tracks are individually determinable in succession and wherein the determined changes of all second conductor tracks are convertible in the single analog-to-digital converter (ADC).

17. The pressure sensor unit as claimed in claim 16, wherein the at least one counter unit is software controlled.

18. The pressure sensor unit as claimed in claim 16, wherein the at least one counter unit and the at least one first and/or second switch unit are arranged on a flexprint or a series of flexprints connected to one another.

19. The pressure sensor unit as claimed in claim 16, wherein two counter units are provided, wherein a first counter unit of these counter units cycles through the lines and a second counter unit of these counter unit cycles through the columns.

20. The pressure sensor unit as claimed in claim 16, wherein the pressure sensor sheet comprises at least one sensor, preferably a temperature sensor or a moisture sensor, and wherein the pressure sensor unit determines sensor data of the at least one sensor.
